(19) United States
(12) Patent Application Publication
(10) Pub. No.: US 2020/0337794 A1
(43) Pub. Date: Oct. 29, 2020

(54) Remote Healthcare-Monitoring System With User Enabled Diagnostic Capabilities

(71) Applicant: Medic LLC, Provo, UT (US)

(72) Inventors: David R. Hall, Provo, UT (US); Conrad Rosenbrock, Provo, UT (US); Ben Swenson, Lehi, UT (US); Jared Eggett, Lehi, UT (US)

(73) Assignee: Jeff Duncan, Provo, UT (US)

(21) Appl. No.: 16/962,983

(22) PCT Filed: Jan. 17, 2018
(86) PCT No.: PCT/US18/14113

§ 371 Date: Jul. 17, 2020

(51) Int. Cl. A61B 50/30; G16H 50/20; G16H 40/67; A61B 90/00; A61B 34/00; A61B 5/0205; A61B 90/96; G06K 9/00

(52) U.S. Cl. CPC A61B 50/30; G16H 50/20; G16H 40/67; A61B 90/361; A61B 5/022; A61B 5/02055; A61B 90/96; G06K 9/00671; A61B 34/25

(57) ABSTRACT

A healthcare-monitoring system collects health data through sensors then analyzes the data. The system may then instruct the user to retrieve one or more medical devices or tests stored within the system and perform an additional diagnostic measurement or test. Medication may also be stored within the system. The location of a medical device, test, or medication may be tracked in relation to the position of the user and the user's body parts. Correct use of the medical device or test and consumption of the medication may be assessed. A user interface may provide instruction for proper use of a medical device or test as well as its location within the system. Sensors may indicate whether a medical device has been returned to its proper place or if a consumable medical product has been removed from storage. The system may automatically reorder consumable medical supplies and track drug compliance.

### CROSS-REFERENCE TO RELATED APPLICATIONS

This application claims priority to PCT International Patent Application Number PCT/US2018/014113 titled “Remote Healthcare-Monitoring System With User Enabled Diagnostic Capabilities” filed on 17 Jan. 2018, which is incorporated herein by reference in its entirety.

### TECHNICAL FIELD

This disclosure relates to diagnostic devices and telemedicine services and uses thereof.

### BACKGROUND

Many medical devices and diagnostic assays typically performed in a clinical setting by trained healthcare professionals are simple enough that a patient could perform them at home with a small amount of training or guidance. However, without training or guidance, many users may operate the medical device or conduct the diagnostic assay incorrectly. This error may render the data collected useless or worse, harmful if the error is not detected.

An option is to visit a healthcare facility to have diagnostic services performed by a trained professional. However, some users may find it inconvenient or impossible to make frequent visits to a healthcare facility. Particularly when the diagnostic metric is relatively simple to collect with some basic instruction, it may seem an inefficient use of time to acquire such healthcare services, even when the results of a late or improper diagnosis are severe. Consequently, these users may go without the full benefit of certain diagnostic tools which could provide a significant and positive impact on their health.

Another problem for those unable to visit a healthcare facility in person is that some metrics are difficult to track without interaction with the patient. Examples include behavior, mobility patterns, and generally how well the patient appears. In some instances, daily interaction is optimal to properly assess a patient's health status. This is impractical for many people.

In addition, drug compliance is a problem for many individuals. More specifically, some people forget to take their medication while some take it more often than it is prescribed or off schedule. Other's may consume outdated medication or medication not meant for them. For some people, unless the patient is consuming the medication in a healthcare facility under the direct supervision of a healthcare provider, proper drug compliance may not occur.

While telemedicine technology has provided remote healthcare services for some situations, it has yet to replace direct interaction between a patient and a trained healthcare provider. Advances are needed to provide remote healthcare services which replace direct human contact.

### SUMMARY

We disclose a healthcare-monitoring system which may use a plurality of sensors, including at least one camera. The sensors may collect diagnostic data and assist a user in utilizing medical devices, tests, and medical products to perform remote healthcare activities. The sensors may perform an initial assessment of a user's health by taking measurements including collecting graphic data from a camera. Algorithms may process the sensor measurements to assess whether additional medical testing or procedures are warranted. The system includes a human-machine interface platform through which the system may instruct the user which medical test or procedure to access and how to use it correctly. The system may include a plurality of units within a container. The needed medical products, tests, or devices may be stored within the units and be easily accessible away from a clinical setting.

The device may include a system core, that includes a graph database, a sensor measurement data stream manager, and an entity tracking system. The graph database may be used to create a differential diagnosis in response to diagnostic data the sensors collect. The entity tracking system may track the location of the medical product, test, or device in real time in relation to the user and the user's body parts. Accordingly, algorithms may use this information to determine whether the user is properly operating the medical device or test. The entity tracking system may also track the user's consumption of medication to improve drug compliance.

The sensors may detect the presence, absence, or reduction of an item in a unit within the container. The sensor may also send a signal to the controller which may transmit a request to a supplier to ship a replacement product. Additionally, the system may measure the gradual reduction of a medication supply to assess whether the user has been compliant in taking the medication as instructed and has taken the correct medication.

### BRIEF DESCRIPTION OF THE DRAWINGS

Non-limiting and non-exhaustive implementations of the disclosure are described with reference to the following figures, wherein like reference numerals refer to like parts throughout the various views unless otherwise specified. Advantages of the disclosure will become better understood regarding the following description and accompanying drawings where:

FIG. 1A is a plan drawing of a user standing before an embodiment of the disclosed healthcare-monitoring system.

FIG. 1B is a perspective drawing of an embodiment of the disclosed healthcare-monitoring system with the cover displaced and showing the units.

FIG. 1C is a perspective drawing of an embodiment of the disclosed healthcare-monitoring system in use by a user.

FIG. 2A is a perspective drawing of an embodiment of the disclosed healthcare-monitoring system showing data being transmitted wirelessly from a device to a remote database.

FIG. 2B is a perspective drawing of the embodiment of FIG. 2A showing data being transmitted wirelessly from the remote database to a device.

FIG. 3 is a perspective drawing illustrating tracking of a medical device which was retrieved from a unit within a device.

FIG. 4A is a perspective drawing of a unit within an embodiment of the disclosed healthcare-monitoring system in which a sensor detects the presence of a medical product.

FIG. 4B is a perspective drawing of the unit of FIG. 4A in which the medical product has been removed from the unit.

FIG. 5 is a perspective drawing of an embodiment of the disclosed healthcare monitoring system in which a user interacts through a mobile communication device.

### DETAILED DESCRIPTION

### Definitions

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure, and are not meant to be limiting in any fashion. Nor do these phrases indicate any kind of preference for the disclosed embodiment.

As used herein “mobile communication device” means a portable computing device which provides wired or wireless communication. Examples include smartphones, tablets, and laptop computers.

As used herein “user” means an individual, human or animal, who interacts with the disclosed healthcare-monitoring system to receive a health analysis, a medical product, or use a diagnostic device or test.

As used herein “electronic communication” means a communication between electronic devices which may be either through wired or wireless methods. In an example, “electronic communication” includes a communication through WiFi.

### Exemplary Embodiments

While this invention is susceptible of embodiment in many different forms, there are shown in the drawings, which will herein be described in detail, several specific embodiments with the understanding that the present disclosure is to be considered as an exemplification of the principals of the invention and is not intended to limit the invention to the illustrated embodiments.

We disclose a remote healthcare-monitoring system which may be used in conjunction with telemedicine methods. The healthcare-monitoring system may include a health monitoring system as disclosed in International Patent Application No. PCT/US18/13836 filed on Jan. 16, 2018 which is hereby incorporated by reference in its entirety. The remote healthcare-monitoring system disclosed herein (hereinafter “the system”) may include a container for housing a plurality of medical products, medial tests, and diagnostic devices. The container may include a plurality of units, each capable of housing a different medical product, medical test, or diagnostic device. The system also includes a plurality of sensors, which include at least one camera, and a controller. The controller may include the non-transient computer readable media as described in International Patent Application No. PCT/US17/63917 filed on Nov. 30, 2017 which is hereby incorporated by reference in its entirety. The material disclosed in PCT/US17/63917 may be used within the system disclosed herein to track medical devices, medical tests, and medical products (including medications) during their use. The application of the matter disclosed in PCT/US17/63917 may further be used to track the locations of items stored in the units described herein similar to the user of the same to track inventory and merchandise placement using, in part, an entity tracking system. The use within the context of inventory and merchandise placement is disclosed in International Patent Application No. PCT/US18/14003 filed on Jan. 17, 2018 which is herein incorporated by reference in its entirety. In the disclosed system, the sensors, which include at least one camera, identify an object which is tagged with a two-dimensional barcode, a tag that emits electromagnetic signals (for example, RFID tags), or other tags known in the art. The sensors within the system may read the tags and identify the position of the item in real time. Algorithms which are the same or similar to facial recognition technology may identify the position of the user and the user's specific body parts. Accordingly, the system may track the position of the item retrieved from the unit within the container in relation to the user to determine correct or incorrect use of the item. In addition, the device may include a perspective camera, a gyrometer, or accelerometer to provide further information about its location in real time.

The system may include a light source directed toward the user and within a field of view of the at least one camera. Different light sources may be used for different purposes. In an example, these light sources may emit one or more of visible light, high color temperature light, infrared light, structured light, and modulated light.

Within the disclosed system, the diagnostic system disclosed in PCT/US18/13836 may assess the user's health and then identify an additional diagnostic metric to further identify any health problems. The diagnostic devices may include otoscope, an ophthalmoscope, an endoscope, a laparoscope, a laryngoscope, a colposcope, a hysteroscope, a bronchoscope, a pharyngoscope, a laparoscope, a dental tool, a stethoscope, a blood pressure cuff, a pulse oximeter, a thermometer, a respirometer, or other medical device known in the art. Other diagnostic devices which may be included in the system include polarization and Raman spectroscopy modules. These devices can be used to measure concentrations of certain nutrients and vitamins, or be used in further correlations for health and disease.

Medical tests that may be included in the units include, but are not limited to, blood or urine glucose monitors, pregnancy tests, ovulation tests, fecal occult tests, urine pH assays or other diagnostic assays known in the art.

Medical products that may be included in the units include, but are not limited to, medications, bandages, topical cremes, joint wraps, and antiseptics.

The device and its accompanying algorithms disclosed in PCT/US17/63917 may be used to assess the user's health as the user stands before a human-machine interface platform and to compile a differential diagnosis. The human-machine interface platform may be a cover which covers the container. In an example, the cover is similar to a mirror placed over a traditional bathroom medicine cabinet. In some embodiments, the cover is a partially silvered mirror. A camera may be placed behind the cover or the mirror, and directed towards a user. In an example, the user may stand in front of the partially silvered mirror as one would stand before a traditional bathroom medicine cabinet. The camera may collect graphical information about the user either through the mirror or through a window disposed in the cover and between the camera lens and the user. In some embodiments, a controller within the system includes algorithms which obfuscate the graphical data for purposes of privacy, safety, and security.

In another embodiment, the container resembles a fishing tackle box and includes a cover that may lift up to expose the units within. This embodiment is a more mobile embodiment and may be used by military or emergency personnel in the field. A mobile communication device may be connected to a controller within the system and function as a human-machine interface platform.

One the algorithms within the system have collected health data from a user and compiled a differential diagnosis, the algorithms identify one or more suggested medical product, test, or device to the user. The one or more suggested medical product, test, or device may be housed within the container and the list communicated to the user through the human-machine interface. Instructions for using the suggested medical product, test, or device may be communicated to the user through the human-machine interface. The human-machine interface may track the suggested medical product, test, or device during use or consumption and provide further instruction to the user as needed to prevent improper use of the medical product, test, or device. These instructions may be provided in real time as the user manipulates the suggested medical product, test, or device so that the item is properly employed. Corrections may be provided should the user deviate from proper use.

The human-machine interface may include options for the user to communicate questions. The human-machine interface may include a touch screen, animated display, a graphical display, a textual display, and may indicate the location of the item stored in a unit within the container. In some embodiments, an image of the item stored in a unit may appear on the human-machine interface in a location on the cover that covers the unit housing the item. The user is thereby instructed where to look for the item.

In addition to diagnostic medical devices housed within the units within the container, the system human-machine interface may be adapted to display a visual or audible sensory stimulus. The cameras and other sensors may track the user's response to the stimulus. For example, the system may emit a sound and track whether the user responds to the sound, or whether and how fast the user turns toward the source of the sound to assess the user's hearing and reaction time. In another example, the human-machine interface may display a moving image and the cameras may track how well and how fast the user's eyes follow the image. Examining the trend of this response time over a period of months or years could identify problems with ocular accommodation as well as neurological issues. The system may show text on the human-machine interface. The human-machine interface could reduce the text size in response to the reaction the camera detects in the user's eyes, similar to reading a traditional eye chart. If the font size is too small for the user's eyes to read, the user's eyes will strain in reading the text and the camera will detect the strain. The light source, for example, light strips along the edge of the cover, may be used in conjunction with cameras to perform ellipsometry on the patient's eye surface. In some embodiments, the light source comprises a plurality of LED lights which are modulated in a time-dependent manner. Each LED reflects off a user's eye at a different angle relative to the camera monitoring that wavelength of light. This allows the shape of the eye, as well as cuts and other aberrations to be detected. The system may provide a bright light which is directed to the user's eyes and assess the ability of the user's pupils to dilate in response to the light emitted by the light source.

The disclosed system may detect the presence, absence, or the reduction of an item in a unit within the container. A sensor, for example a scale or other type of pressure sensor, may conduct a status measurement to detect whether or not the item has been removed from the unit. For items that are consumable in portions, for example bandages, antiseptics, and other medications, the change in the amount of the item may be detected. The status measurement may then be transmitted to the controller for processing by an algorithm stored therein or to a remote database.

In an example, the system may instruct the user to retrieve an antiseptic solution and a bandage from the units. The user may do so but use only a portion of each. The user may return the remaining portion of the antiseptic solution and package of bandages to their respective units. The sensor may detect the change in the contents of the bottle of antiseptic solution and the package of bandages by measuring a change, for example, in mass. The same may be done for a bottle of pills. The user may take the bottle from a unit, retrieve one pill, then return the bottle minus the one pill. The reduction in the amount of the medication may be measured and the data processed for uses that include monitoring drug compliance.

The system may use the status measurements to track the user's compliance with the instructions the system provides. The system may also use the status measurements to automatically order replacement products. For example, algorithms within the system may provide instructions to the controller to send an electronic signal to a supplier containing a request to ship a replacement product when the status measurement indicates that the item is absent or that the item has reached a defined minimum.

The system may be used to encourage and track drug compliance. As described above, the system may monitor the amount and position of each medication housed within the container. Cameras may detect tags, for example two-dimensional barcodes, on the medication packaging. Status measurements may monitor changes in the amount of medication as describe above and the camera may record the user consuming the mediation.

Facial recognition technology may confirm that the user to whom the medication was prescribed was the user who actually consumed the medication. This may prevent, or at least identify, when an individual attempts to consume another person's medication. Similarly, a user who accidently attempts to consume the wrong medication could be stopped by providing a warning. Should the user continue despite warnings, the mistake would be known should there be adverse events.

The facial recognition technology may also be used to provide access to the medication only to the user to whom the medication was prescribed. Optionally, an emergency protocol may be provided to allow caregivers to access the user's medication should the user be unable to access the medication during a medical emergency.

In some embodiments, the system includes a microphone and speaker system. This allows the user to interact with smart apps such as video calling apps, voice dictation, media players. A user may also communicate remotely with a healthcare provider using a method that resembles a telephone call or video conference. Additionally, the microphones together with facial, eye and other features the camera detects, may assist in diagnosing depression and other mental illnesses.

As mentioned, the one or more camera may include a video camera. The video camera may record the user's movements, behavior, and interactions with the human-machine interface. This data may be used to diagnose autism, attention deficit disorder (ADD), attention deficit hyperactivity disorder (ADHD), and other neurological, psychological, and neuropsychological disorders.

The microphone, speaker system, and recorded graphic data may be used in combination to provide medical intervention in the case of a medical emergency. Using voice commands, the system may alert first responders and inform them of the user's condition and events leading to it. The human-machine interface may communicate information to the user which may prevent the situation from worsening until first responders arrive.

In some embodiments, sensitive cameras may be included in the system which enable the system to perform thin film interference techniques. These techniques could be used to detect the presence and thickness of oily layers on a user's skin. Knowing the location and size of oil patches may be used to suggest cosmetic or skin care products and where to apply them. Color and condition of the skin can also be included in the recommendation engine. Changes in these variables over time can highlight side effects of cosmetic and skin care product use or potential benefits that are presently unknown.

Alternatively, the system could include an apparatus that directly contacts the user's face to detect texture, color and oiliness. For example, the apparatus may be a mask or a device which the user swipes across areas of the face much like a temporal scanner thermometer.

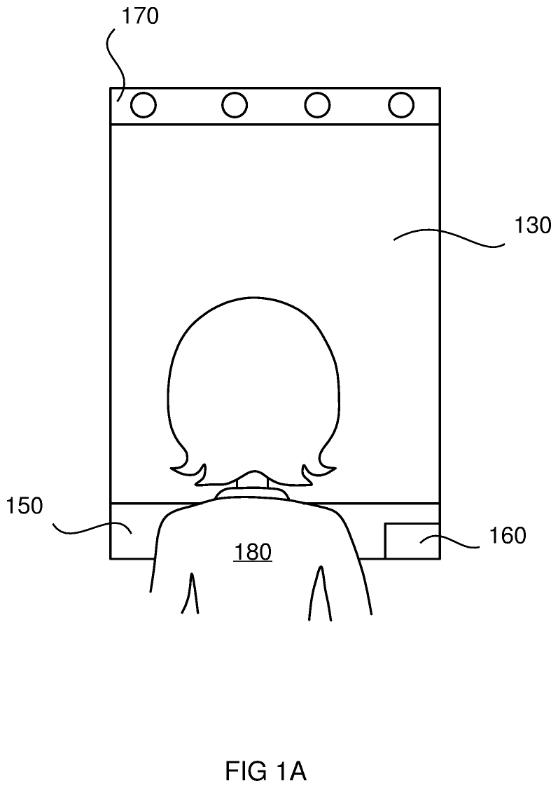

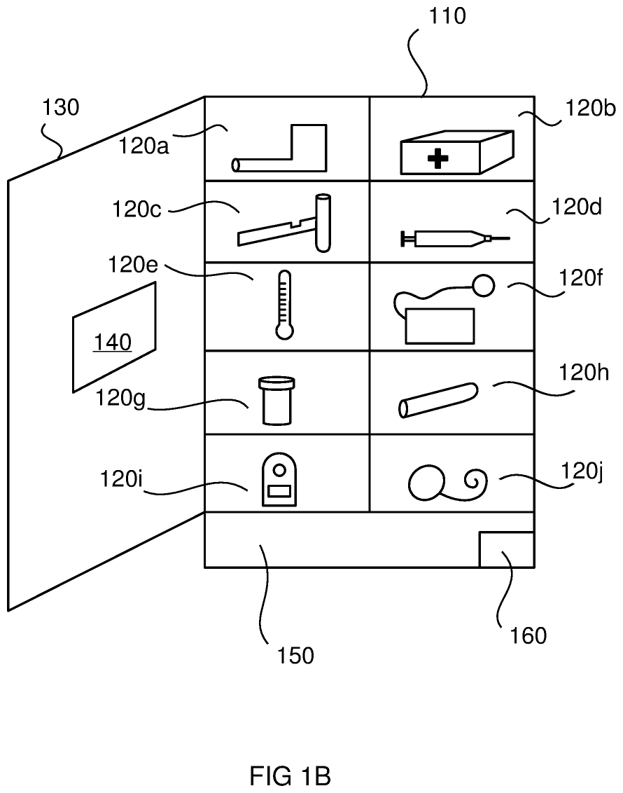

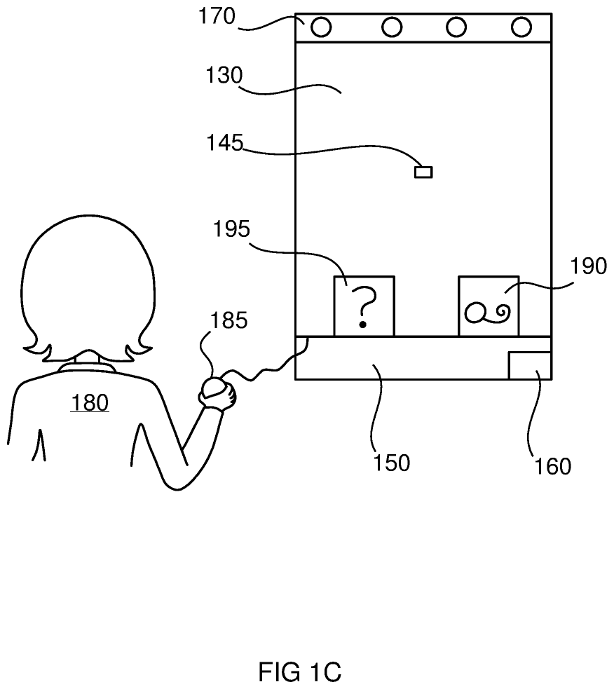

Referring now to the drawings, FIG. 1A illustrates a device which is part of an embodiment of the disclosed healthcare-monitoring system. The device includes cover 130 which may be a partially silvered mirror. Controller 150 is connected to cover 130 and includes communication port 160. Light source 170 directs light toward user 180.

FIG. 1B illustrates the device of FIG. 1A with cover 130 in a position which exposes units 120a-j within container 110. Each of units 120a-j houses a medical product, a medial test, or a diagnostic device. For example, unit 120a houses a respirometer, unit 120b houses first aid supplies, unit 120c houses an ophthalmoscope, unit 120d houses a syringe, unit 120e houses a thermometer, unit 120f houses a blood pressure cuff, unit 120g houses a bottle of medication, unit 120h houses a roll of bandages, unit 120i houses a glucose monitor, and unit 120j houses a stethoscope. The stethoscope of unit 120j may be connected to controller 150 so that the user's heart sounds may be stored in a memory of controller 120j. Camera 140 is disposed on a back side of cover 130. The lens of camera 140 is directed toward the position where a user might stand when the user approaches cover 130. In embodiments in which cover 130 is a partially silvered mirror, camera 140 may collect graphic data of the user and the user's activities through cover 130.

FIG. 1C illustrates user 180 after camera 140 has collected graphic data and proposed that the user retrieve a stethoscope 185 from a unit behind cover 130 and to collect cardiac data. In this embodiment, camera 140 retrieves the graphic data through a window 145 so that cover 130 does not inhibit the camera's view. User 180 has retrieved stethoscope 185 and connected it to controller 150. A human-machine interface has provided two images which are visible on cover 130. Image 190 shows an image of stethoscope 190 in front of the unit in which it was housed. Image 195 shows an image of a question mark. In this embodiment, cover 130 includes a touchscreen. A user may have been instructed to touch an image showing a question mark which is next to the image of the item for which the user requires instruction. In FIG. 1C, the user may touch image 195 to receive instruction for use of the device shown in image 190, in this case, stethoscope 185.

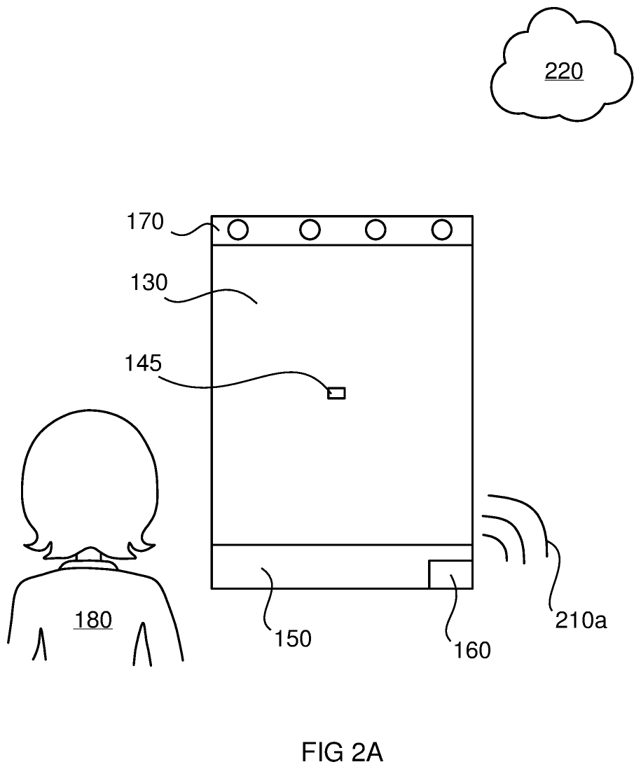

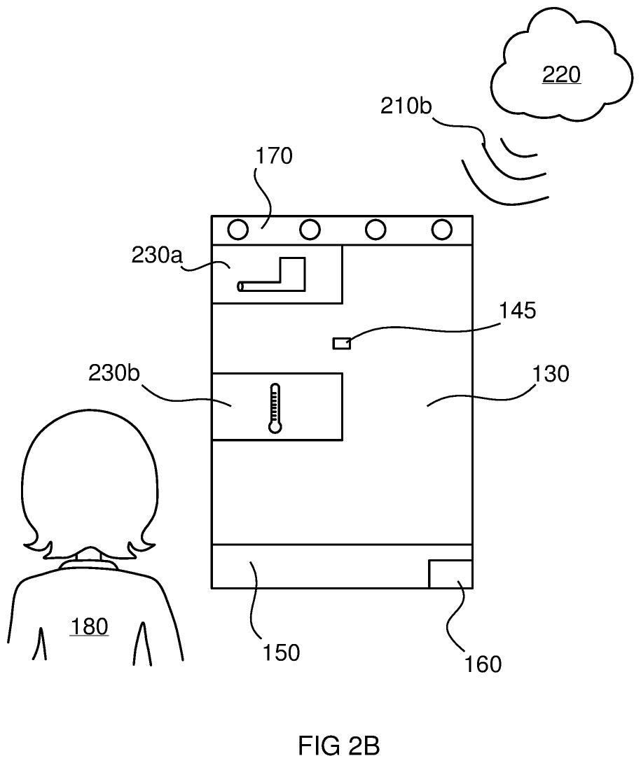

As illustrated in FIG. 2A user 180 has approached cover 130. Graphic data comprising data associated with the health of user 180 has been collected has been collected by a camera through window 145. The graphic data is being transmitted to remote database 220 through communication port 160. In this embodiment, the graphic data is transmitted through wireless signal 210a.

As illustrated in FIG. 2B, the graphic data transmitted using wireless signal 210a in FIG. 2A has been processed using algorithms stored on remote database 220. The algorithms propose which suggested medical product, test, or device the user needs to collect additional health data based on the graphic data the camera collected. Wireless signal 210b transmits information to controller 150 suggesting the user collect data using a respirometer and a thermometer. A human-machine interface presented on cover 130 shows image 230a and image 230b. Image 230a shows an image of a respirometer at a location on cover 130 which is adjacent to the unit housing a respirometer. Image 230b shows an image of a thermometer at a location on cover 130 which is adjacent to the unit housing a thermometer.

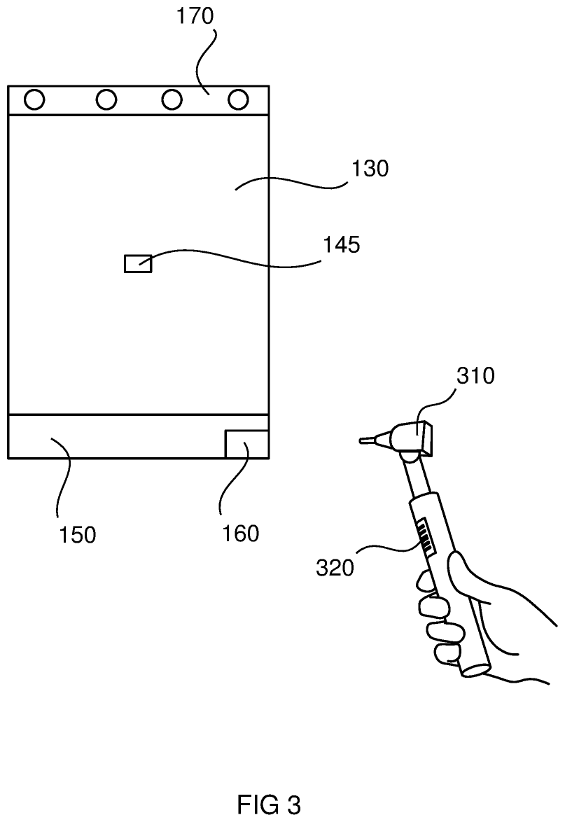

FIG. 3 illustrates a user holding otoscope 310 which the user has retrieved from a unit behind cover 130. Otoscope 310 has been tagged with bar code 320. A bar code reader, which may be a camera behind cover 130, reads bar code 320 through window 145. As the user moves otoscope 310 during use, the bar code reader tracks the location of otoscope 310. Sensors may also track the user and the user's body parts using an entity tracking system. The healthcare-monitoring system thereby assesses proper or improper use of otoscope 310.

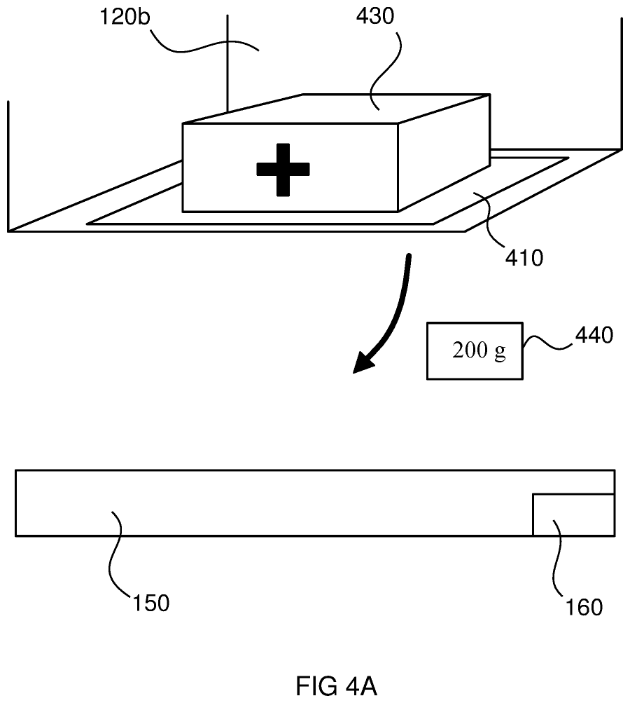

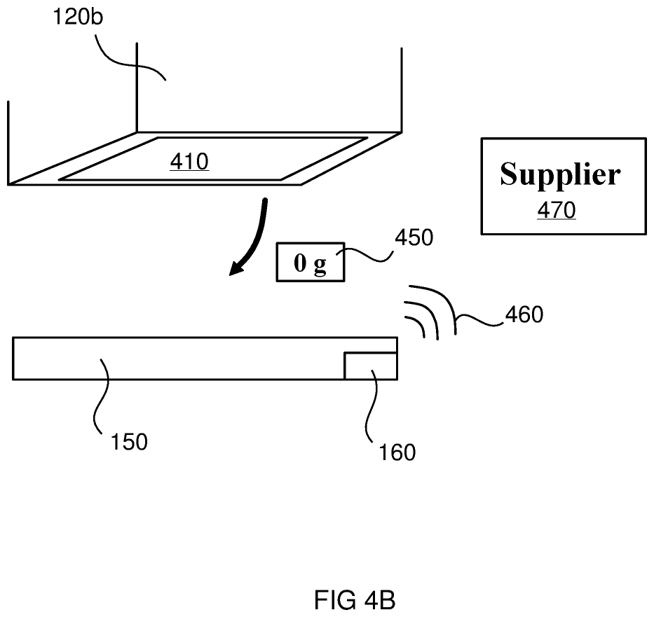

FIG. 4A shows a close-up view of an embodiment of a unit within a container. Pressure sensor 410 is disposed on the bottom surface of unit 120b. First aid supply kit 430 is stored within unit 120b and rests on pressure sensor 410. According to FIG. 4A, pressure sensor 410 collects status measurement 440 which indicates that current mass of first aid supply kit 430 is 200 g. Pressure sensor 410 is in electronic connection with controller 150 and transmits status measurement 440 to controller 150 as indicated by the solid arrow.

FIG. 4B again shows unit 120b as originally presented in FIG. 4A. However, first aid supply kit 410 has been removed from unit 120b, perhaps having been consumed by a user. Pressure sensor 410 collects status measurement 450 which indicates a mass of 0 g rests on pressure sensor 410. Pressure sensor 410 transmits status measurement 450 to controller 150. Instructions stored in a memory on controller 150 cause controller 150 to send a request for a replacement supply of first aid supply kit 430. This request is transmitted through communication port 160 through wireless signal 460 to supplier 470. Consequently, first aid supply kit 430 is automatically reordered each time its mass falls to 0 g or below a defined mass (prior to complete absence of the supply).

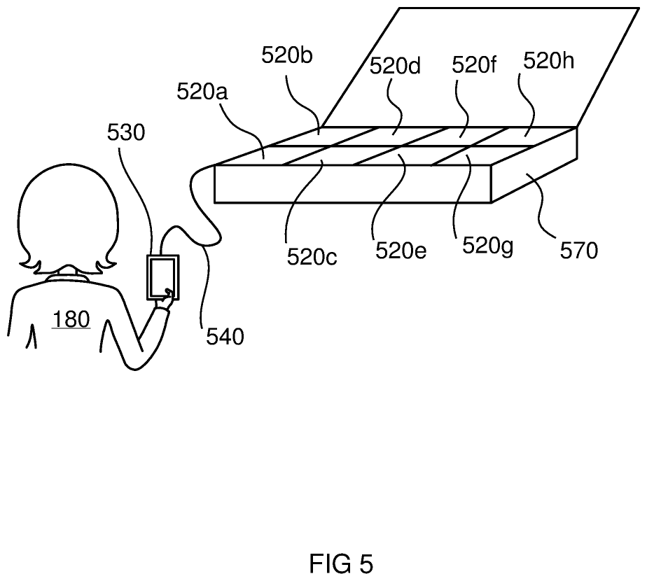

FIG. 5 illustrates another embodiment of a device which may be a part of the disclosed healthcare-monitoring system. Container 510 includes units 520a-h. Similar to container 110 previously shown, container 510 houses a medical product, test, or device in each of units 520a-h. In contrast to container 110 which resembles a bathroom medicine cabinet, container 510 is more portable and may be used in remote locations, for example, by military personnel or first responders in the field. User 180 is shown holding mobile communication device 530 which is connected to container 510 through wired connection 540. A screen on mobile communication device 530 displays the human-machine interface, similar to cover 130 as shown in FIGS. 1C and 2B. A camera within mobile communication device 530 may collect graphic data analogous to camera 140 shown in FIG. 1B. A controller may be contained within container 510. Alternatively or in addition, compute resource housed in mobile communication device 530 may store and transmit data and run algorithms to process data.

While specific embodiments have been illustrated and described above, it is to be understood that the disclosure provided is not limited to the precise configuration, steps, and components disclosed. Various modifications, changes, and variations apparent to those of skill in the art may be made in the arrangement, operation, and details of the methods and systems disclosed, with the aid of the present disclosure.

Without further elaboration, it is believed that one skilled in the art can use the preceding description to utilize the present disclosure to its fullest extent. The examples and embodiments disclosed herein are to be construed as merely illustrative and exemplary and not a limitation of the scope of the present disclosure in any way. It will be apparent to those having skill in the art that changes may be made to the details of the above-described embodiments without departing from the underlying principles of the disclosure herein.

We claim:

1. A healthcare-monitoring system comprising:
a container comprising:
a plurality of units, each of the plurality of units housing at least one medical product, test, or device;
a plurality of sensors comprising at least one camera; and
a human-machine interface platform;
a controller comprising a memory and a communication port, wherein the controller is in electronic communication with the at least one camera and with the human-machine interface platform, and wherein the controller receives graphic data from the camera;
non-transitory computer-readable media comprising a first set of instructions for assessing data collected by the sensors, creating a differential diagnosis, and providing a list of at least one suggested medical product, test, or device;
non-transitory computer-readable media comprising a second set of instructions for displaying material a human-machine interface, wherein the human-machine interface identifies at least one of the plurality of units which houses at least one suggested medical product, test, or device and provides guidance for the user of each of the at least one suggested medical product, test, or device; and
a system core, comprising:
a graph database;
a sensor measurement data stream manager; and
an entity tracking system, wherein the entity tracking system tracks the at least one suggested medical product, test, or device and its location in a time dependent manner, and wherein the system core is in electronic connection with each of the plurality of sensors.

2. The healthcare-monitoring system of claim 1, wherein the controller further comprises non-transitory computer-readable media comprising a third set of instructions for applying an algorithm to transform the graphic data into obfuscated graphic data, to create an analysis of the obfuscated graphic data, and to create an assessment of the user's health status.

3. The healthcare-monitoring system of claim 2, wherein the third set of instructions includes directions to transmit the obfuscated graphic data through the communication port to a remote database.

4. The healthcare-monitoring system of claim 3, wherein the non-transitory computer-readable media comprising the first set of instructions is stored within the remote database.

5. The healthcare-monitoring system of claim 1, wherein the camera is adapted to track a location of the at least one suggested medical product, test, or device during use; wherein the non-transitory computer readable media comprises instructions for identifying a correct use of the at least one suggested medical product, test or device and instructions for transmitting a signal through the human-machine interface, and wherein the signal provides guidance for the correct use of the at least one suggested medical product, test, or device.

6. The healthcare-monitoring system of claim 1, wherein the cover comprises a partially silvered mirror, and wherein the camera is disposed behind the partially silvered mirror.

7. The healthcare-monitoring system of claim 1, wherein the at least one camera comprises at least one of a 3D time of flight camera, a stereoscopic camera, an infrared thermal imaging camera, a video camera, a structured light 3D scanner, and a still image camera.

8. The healthcare-monitoring system of claim 1, further comprising a light source, wherein the light source is positioned to emit light toward an angle of view of the camera.

9. The healthcare-monitoring system of claim 8, wherein the light source emits one or more of visible light, high color temperature light, infrared light, structured light, and modulated light.

10. The healthcare-monitoring system of claim 1, wherein the at least one medical product, test, or device comprises an otoscope, an ophthalmoscope, an endoscope, a laparoscope, a laryngoscope, a colposcope, a hysteroscope, a bronchoscope, a pharyngoscope, a laparoscope, a dental tool, a stethoscope, a blood pressure cuff, a pulse oximeter, a thermometer, a respirometer, a blood or urine glucose monitor, a pregnancy test, an ovulation test, a fecal occult test, a urine pH assay, medications, bandages, topical cremes, and antiseptics.

11. The healthcare-monitoring system of claim 1, wherein the at least one medical product, test, or device comprises a medication.

12. The healthcare-monitoring system of claim 1, wherein each of the at least one medical product, test, or device comprises a two-dimensional barcode, wherein the camera is adapted to collect data comprising the two-dimensional barcode, and wherein the entity tracking system is adapted to track the location of the at least one medical product, test, or device by tracking a location of the two-dimensional barcode.

13. The healthcare-monitoring system of claim 12, wherein the at least one medical product, test, or device comprises a medication.

14. The healthcare-monitoring system of claim 1, wherein one of the plurality of sensors comprises a reader adapted to detect an electromagnetic signal, wherein each of the at least one medical product, test, or device comprises a tag adapted to emit the electromagnetic signal, and wherein the entity tracking system is adapted to track the location of the at least one medical product, test, or device by tracking a location of the tag.

15. The healthcare-monitoring system of claim 1, wherein the human-machine interface platform comprises a cover disposed over the plurality of units.

16. The healthcare-monitoring system of claim 15, wherein the cover comprises a partially silvered mirror, wherein the camera is disposed between the partially silvered mirror and the plurality of units.

17. The healthcare-monitoring system of claim 15, wherein the cover comprises a touch screen.

18. The healthcare-monitoring system of claim 1, wherein the human-machine interface platform comprises a mobile communication device.

19. The healthcare-monitoring system of claim 1, wherein the at least one medical product, test, or device comprises at least one of a perspective camera, an accelerometer, and a gyrometer.

20. The healthcare-monitoring system of claim 1, wherein the human-machine interface displays graphical images of a proper use of the at least one suggested medical product, test, or device.

21. The healthcare-monitoring system of claim 1, further comprising an inventory sensor in at least one of the plurality of units, wherein the inventory sensor is configured to detect the presence, reduction, or absence of at least one of the at least one medical product, test, or device and to transmit a status signal to the controller, wherein the status signal indicates the presence, reduction, or absence of the at least one medical product, test, or device.

22. The healthcare-monitoring system of claim 21, wherein the at least one medical product, test, or device comprises a medication.

23. The healthcare-monitoring system of claim 21, wherein inventory sensor is in electronic connection with the controller, and wherein a memory within the controller comprises non-transitory computer-readable media comprising a fourth set of instructions, wherein the fourth set of instruction comprise directions for transmitting a request to a supplier for a replacement when the controller receives a status signal indicating the reduction or absence of the at least one medical product, test, or device.

24. The healthcare-monitoring system of claim 1, wherein the human-machine interface is adapted to display a visual or audible sensory stimulus, and wherein the at least one camera is adapted to collect graphic data representing the user's response to the stimulus.
